(19) United States
(12) Patent Application Publication
(10) Pub. No.: US 2023/0238115 A1
(43) Pub. Date: Jul. 27, 2023

(54) MEDICAL INFORMATION SYSTEM, CHARGE CALCULATING DEVICE, AND CHARGE CALCULATING METHOD

(71) Applicant: SONY GROUP CORPORATION, TOKYO (JP)

(72) Inventor: MASAYA TAKEMOTO, TOKYO (JP)

(21) Appl. No.: 18/001,036

(22) PCT Filed: May 31, 2021
(86) PCT No.: PCT/JP2021/020666

§ 371 Date: Dec. 7, 2022

(30) Foreign Application Priority Data
Jun. 16, 2020 (JP) 2020-103945

(51) Int. Cl. G16H 30/20; A61B 5/00

(52) U.S. Cl. CPC G16H 30/20; A61B 5/0013

(57) ABSTRACT

Costs for a system change are inhibited. A medical information system according to an embodiment includes: a first medical device (101A) that acquires a medical image during surgery; an edge server (300) that processes the medical image acquired by the first medical device; and a second medical device (101B) that displays the medical image that has been processed by the edge server. The edge server includes: a framework (301) including one or more applications that process the medical image; and a charge calculator (303) that calculates a charge in accordance with a situation of use of the one or more applications for the medical image.

### FIELD

The present disclosure relates to a medical information system, a charge calculating device, and a charge calculating method.

### BACKGROUND

There have been developed medical information systems that perform image processing with a large calculation amount on an external calculation processing device connected to a medical device such as a server in a hospital. In recent years, a use case in which applications provided from a plurality of companies are operated on one system instead of one company constructing the entire medical information system has been studied. In such a use case, functions required by a user can be provided to a server in a flexible combination of each other in a form of applications, instead of the functions being provided to the user in a form in which the functions are incorporated in hardware as in a conventional manner.

### CITATION LIST

### Patent Literature

Patent Literature 1: Japanese Patent No. 6466060

### SUMMARY

### Technical Problem

In a medical field, it is desired to construct a flexible system in accordance with various needs of users. Conventionally, however, a user has purchased outright a system constructed in accordance with the needs of the user. In that case, once the system is introduced, it is difficult to flexibly change the system in accordance with subsequent business expansion, business change, evolution of medical technology, and the like, which unfortunately generates a lot of costs.

Therefore, the present disclosure proposes a medical information system, a charge calculating device, and a charge calculating method capable of inhibiting costs for a system change.

### Solution to Problem

A medical information system includes an edge server connected to a medical device that acquires a medical image during surgery, wherein the edge server includes: a framework including one or more applications that process the medical image; and a charge calculator that calculates a charge in accordance with a situation of use of the one or more applications for the medical image.

### BRIEF DESCRIPTION OF DRAWINGS

FIG. 1 is a block diagram for outlining a provision model according to a first embodiment.

FIG. 2 is a block diagram illustrating one example of an edge server according to the first embodiment.

FIG. 3 illustrates one example of a surgical procedure table according to the first embodiment.

FIG. 4 illustrates one example of a charge table according to the first embodiment.

FIG. 5 is a block diagram illustrating one example of an edge server according to a variation of the first embodiment.

FIG. 6 is a flowchart illustrating one example of a charge calculating operation according to the first embodiment.

FIG. 7 is a flowchart illustrating one example of a charge calculating operation according to a second embodiment.

FIG. 8 is a flowchart illustrating one example of a charge calculating operation according to a third embodiment.

FIG. 9 is a flowchart illustrating one example of a charge calculating operation according to a fourth embodiment.

FIG. 10 is a flowchart illustrating one example of a charge calculating operation according to a fifth embodiment.

FIG. 11 is a flowchart illustrating one example of a charge calculating operation according to a sixth embodiment.

FIG. 12 is a hardware configuration diagram illustrating one example of a computer that implements a function according to the embodiments.

### DESCRIPTION OF EMBODIMENTS

Preferred embodiments of the present disclosure will be described in detail below with reference to the accompanying drawings. Note that, in the present specification and the drawings, components having substantially the same functional configuration are denoted by the same reference signs, and redundant description thereof will be omitted.

Note that the description will be given in the following order.
1. Introduction
2. First Embodiment
2.1 Outline of Provision Model
2.2 System Configuration of Edge Server
2.2.1 Variation of Edge Server
2.3 Example of Charge Calculating Operation
3. Second Embodiment
3.1 Example of Charge Calculating Operation
4. Third Embodiment
4.1 Example of Charge Calculating Operation
5. Fourth Embodiment
5.1 Example of Charge Calculating Operation
6. Fifth Embodiment
6.1 Example of Charge Calculating Operation
7. Sixth Embodiment
7.1 Example of Charge Calculating Operation
8. Other Embodiments
9. Conclusion
10. Hardware Configuration

### 1. Introduction

As described above, in recent years, a medical information system in which applications provided from a plurality of companies are operated on one system has been studied. Constructing such a system allows the system that has been introduced once to be flexibly changed later, so that a user can easily change the system in accordance with subsequent business expansion, business change, evolution of medical technology, and the like.

For a medical information system in which applications provided from a plurality of companies are operated on one system, not outright purchase type applications but billing type applications are desired to be provided in order to introduce various applications in accordance with the needs of users.

Then, in the following embodiments, there are proposed a medical information system, a charge calculating device, and a charge calculating method capable of providing an application at a charge of use in accordance with a situation of use of a user. This makes it possible to propose a medical information system capable of being flexibly changed after being introduced and inhibiting costs for a system change, a charge calculating device, and a charge calculating method. This also makes it possible to pay a charge in accordance with a situation of use of an application calculated from a billing amount to an application providing company.

### 2. First Embodiment

First, a medical information system, a charge calculating device, and a charge calculating method according to a first embodiment will be described in detail with reference to the drawings.

### 2.1 Outline of Provision Model

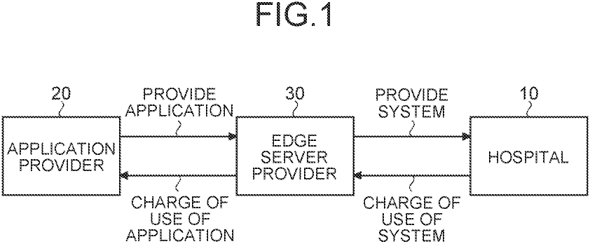

FIG. 1 is a block diagram for outlining a provision model according to the first embodiment. As illustrated in FIG. 1, a hospital 10, an application provider 20, and an edge server provider 30 appear in the provision model according to the embodiment. In the provision model in FIG. 1, the edge server provider 30 receives an application provided from the application provider 20, and the edge server provider 30 constructs an edge server, which is a system mounted with the application. Then, the constructed edge server is delivered to the hospital 10, which is a user.

### (Hospital 10)

The hospital 10 is a user of a medical information system provided by the edge server provider 30. The hospital 10 executes processing such as image processing, analysis, and parsing on various pieces of information (hereinafter, referred to as medical information) by using the medical information system provided by the edge server provider 30. The medical information includes medical images (which may be moving images) acquired by a medical device such as an X-ray diagnostic device, an X-ray CT device, an MRI device, a nuclear medicine diagnostic device, and an ultrasonic diagnostic device, fluorescent signals and sample data acquired by a fluorescent diagnostic device such as a flow cytometer and a fluorescent microscope, and an electronic health record created based on diagnosis of a patient.

### (Application Provider 20)

The application provider 20 is, for example, a developer and a provider of an application. The application provider 20 creates various applications for processing, parsing, analysis, and the like on medical information. The application provider 20 provides the created application to the edge server provider 30 in response to a request from the edge server provider 30 (or hospital 10).

### (Edge Server Provider 30)

The edge server provider 30 constructs an edge server in accordance with the scale of the hospital 10, an introduction purpose, and the like, and delivers the constructed edge server into the hospital 10. The edge server constructed by the edge server provider 30 is only required to be able to execute an application provided from the application provider 20, and may have a functional configuration of a common information processing device. Furthermore, the edge server may be a single server or a server group including a plurality of servers. In the case, part or all of the edge server may be a cloud server. When the part or all is a cloud server, the edge server provider 30 provides access information for accessing the cloud server and the like to the hospital 10.

Moreover, the edge server provider 30 may provide the edge server to the hospital 10 in various modes for each hospital, each site or ward of a hospital, each floor of a ward, and each diagnosis and treatment department of a hospital.

### 2.2 System Configuration of Edge Server

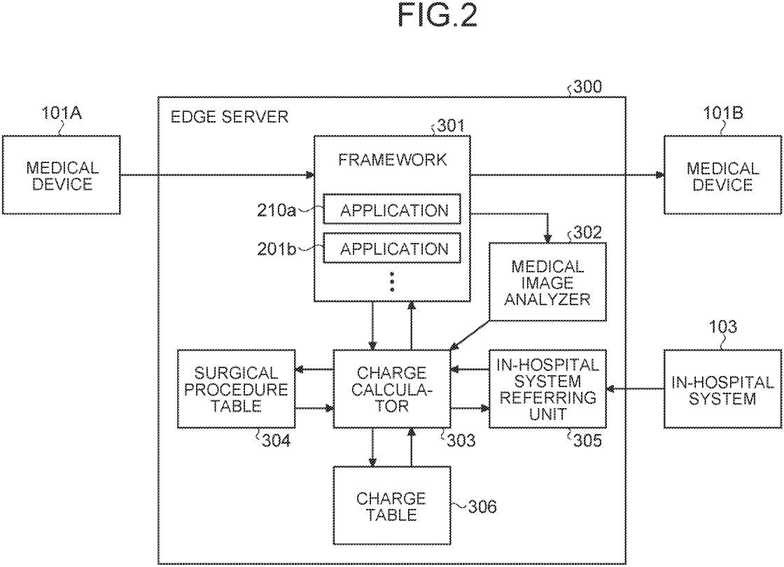

Next, an example of an edge server provided by the edge server provider 30 to the hospital 10 will be described. FIG. 2 is a block diagram illustrating one example of an edge server according to the embodiment. As illustrated in FIG. 2, an edge server 300 includes, for example, a framework 301, a medical image analyzer 302, a charge calculator 303, a surgical procedure table 304, an in-hospital system referring unit 305, and a charge table 306. For example, the medical image analyzer 302, the charge calculator 303, the surgical procedure table 304, the in-hospital system referring unit 305, and the charge table 306 in the edge server 300 constitute a charge calculating device (or system) that calculates a charge of use in accordance with a situation of use of applications 201a, 201b, ... under the management of the framework 301.

### (Framework 301)

The framework 301 constitutes service to be provided to the hospital 10, and is used by being accessed by an operator from a user terminal 102 in the hospital 10. For example, one or more applications 201a, 101b, ... provided from the application provider 20 constitute the framework 301. When one or more applications 201a, 101b, ... are not individually distinguished, the applications are denoted by a reference sign 201 below. Medical information such as a medical image acquired by a medical device 101 in the hospital 10 is input to each application 201 of the framework 301.

An application 201 constituting the framework 301 can be added or replaced at any time. That is, even after the construction of the edge server 300, the application 201 constituting the framework 301 can be changed by a procedure similar to a procedure of installing or uninstalling a common application, for example. This allows the hospital 10, which is a user, to freely change the framework 301 even after the instruction of a medical information system, so that costs for a system change can be inhibited.

Here, the framework 301, which controls a plurality of applications 201, generates information for calculating a charge of use for each application 201 (hereinafter, referred to as application use information) from a situation of use of the application 201 of the hospital 10, which is a user, and transmits the generated application use information to the charge calculator 303. Furthermore, the framework 301 transmits, for example, an image or a moving image input from a medical device 101A at the time of surgery (hereinafter, referred to as surgical image) to the medical image analyzer 302.

### (Medical Image Analyzer 302)

The medical image analyzer 302 generates information for calculating a charge of use for each application 201 (hereinafter, referred to as surgical information) by analyzing a surgical image input from the framework 301, and transmits the generated surgical information to the charge calculator 303.

### (Charge Calculator 303)

The charge calculator 303 calculates a charge of use for each application 201 based on application use information and/or surgical information input from the framework 301 and/or the medical image analyzer 302. For example, the charge calculator 303 calculates the charge of use for each application 201 by referring to the charge table 306 based on the application use information. At that time, the charge calculator 303 may refer to the surgical procedure table 304 based on surgical information input from the medical image analyzer 302, and calculate or correct the charge of use of the application 201 based on the specified surgical procedure. Furthermore, the charge calculator 303 may acquire information on a charging target (e.g., information on diagnosis and treatment department in charge of diagnosis, surgery, and the like of patient and information on presence or absence of counting of medical service fee points) from an in-hospital system 103 installed in the hospital 10, and calculate or correct the charge of use of the application based on the acquired information (hereinafter, referred to as diagnosis and treatment information).

### (Surgical Procedure Table 304)

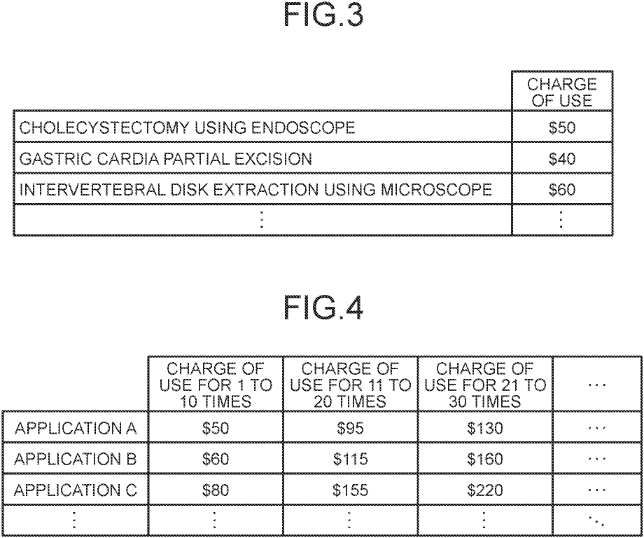

The surgical procedure table 304 holds a charge of use for each surgical procedure of surgery. FIG. 3 illustrates one example of the surgical procedure table 304 according to the embodiment. As illustrated in FIG. 3, a charge of use is associated with each surgical procedure in the surgical procedure table 304. Note that, although a charge of use is set for each surgical procedure in the example, this charge of use may be a charge of use of the application 201 assumed to be used or registered to be used for each surgical procedure. Alternatively, charges of use registered in the surgical procedure table 304 are basic charges, and a charge of use of the application 201 actually used may be added to the charges.

Furthermore, the medical image analyzer 302 may specify a surgical procedure, or the charge calculator 303 may specify the surgical procedure. When the medical image analyzer 302 specifies the surgical procedure, surgical information includes information for identifying a surgical procedure of surgery.

### (In-Hospital System Referring Unit 305)

The in-hospital system referring unit 305 is communicably connected to the in-hospital system 103 via a LAN, a WAN, the Internet, a mobile communication network (including long term evolution (LTE), fifth generation mobile communication system, and the like), or the like, and acquires diagnosis and treatment information from the in-hospital system 103.

### (Charge Table 306)

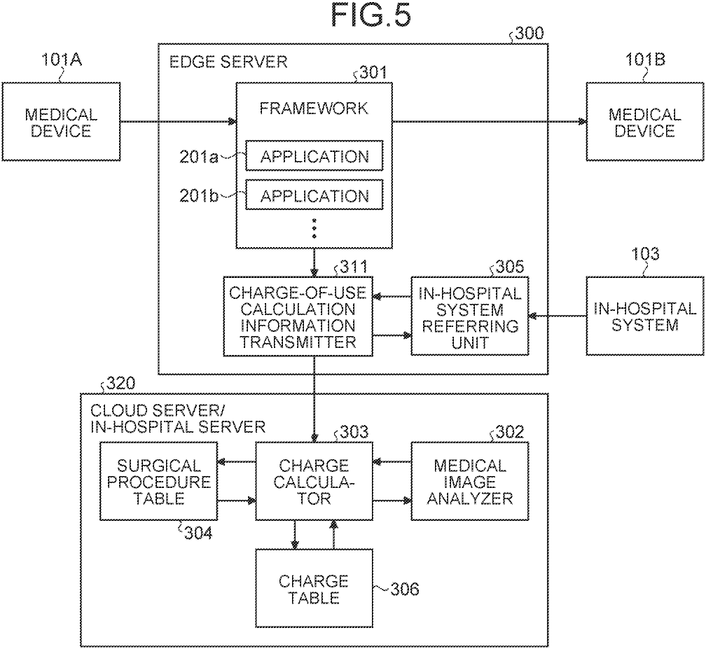

The charge table 306 holds a charge of use for each application 201. FIG. 4 illustrates one example of the charge table 306 according to the embodiment. In the example in FIG. 4, a charge of use in accordance with the number of times of use of each application 201 is registered. Note, however, that the charge table 306 in FIG. 4 is merely one example, and may be variously modified as described later.

As described above, the edge server 300 inputs medical information such as a medical image (which may be a moving image) from the medical device 101A in the hospital 10, performs predetermined processing on the input medical information by using the application 201 provided from the application provider 20, and transmits a processing result obtained by the processing to a medical device 101B of the hospital 10. Note that the medical device 101B may be the same as the medical device 101A, or may be another medical device. Furthermore, the medical device 101B is not limited to a medical device, and may be various devices including a mere display device and an information processing device such as a personal computer.

For example, when the medical device 101A is a camera control unit (CCU) of an endoscope system or the like, the edge server 300 performs image processing on an endoscopic image (medical information) received from the CCU (medical device 101A) by using the application 201, and transmits the result to a monitor (medical device 101B).

### 2.2.1 Variation of Edge Server

Although FIG. 2 illustrates a case where the medical information system is constructed in the single edge server 300, such configuration is not a limitation. For example, as in a variation in FIG. 5, part (or all) of the functions of the edge server 300 in FIG. 2 may be disposed in a cloud server on a network or an in-hospital server in the hospital 10 (hereinafter, referred to as cloud server/in-hospital server 320). In the example in FIG. 5, the medical image analyzer 302, the charge calculator 303, the surgical procedure table 304, the in-hospital system referring unit 305, and the charge table 306 are disposed in the cloud server/in-hospital server 320, and a charge-of-use calculation information transmitter 311 is added to the edge server 300.

The charge-of-use calculation information transmitter 311 transmits application use information and a surgical image input from the framework 301 and diagnosis and treatment information input from the in-hospital system referring unit 305 to the charge calculator 303 of the cloud server/in-hospital server 320.

### 2.3 Example of Charge Calculating Operation

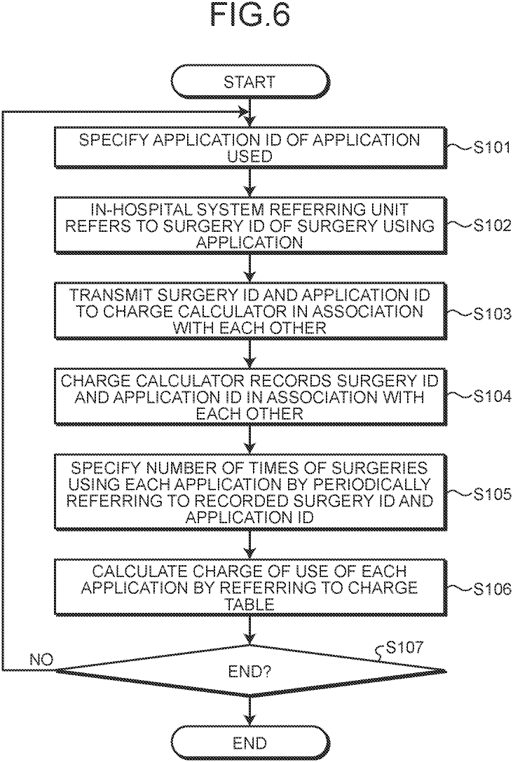

Subsequently, an example of a charge calculating operation according to the embodiment will be described. FIG. 6 is a flowchart illustrating one example of the charge calculating operation according to the embodiment. Note that, although, in the following description, a case where a charge generated when a medical information system is used during surgery is calculated will be illustrated, this is not a limitation. A charge generated when the medical information system is used for an image or a moving image recorded during surgery or sample data acquired at the time of parsing or analyzing a tissue sample can also be calculated.

As illustrated in FIG. 6, during surgery in the hospital 10, in the operation, first, the framework 301 of the edge server 300 specifies identification information for identifying the application 201 used during the surgery (hereinafter, referred to as application ID) (Step S101). The framework 301 transmits the specified application ID to the charge calculator 303.

Next, the in-hospital system referring unit 305 acquires identification information for specifying surgery using the application 201 corresponding to the specified application ID (hereinafter, referred to as surgery ID) by referring to the in-hospital system 103 (Step S102). Note that the in-hospital system 103 manages application IDs for specifying the application 201 used for each surgery.

Then, the in-hospital system referring unit 305 transmits the acquired surgery ID and application ID to the charge calculator 303 in association with each other (Step S103) .

The charge calculator 303 records the received surgery ID and application ID in a predetermined recording device in association with each other (Step S104).

Thereafter, the charge calculator 303 specifies the number of times of surgeries using each application 201 by periodically referring to the recorded surgery ID and application ID (Step S105). Note that the term “periodically” may be, for example, monthly, weekly, or daily.

Then, the charge calculator 303 calculates a charge of use in accordance with the number of times of use of each application 201 in the periodic period by referring to the charge table 306 (Step S106).

Thereafter, the edge server 300 determines whether or not to end the operation (Step S107). When determining to end the operation (YES in Step S107), the edge server 300 ends the operation. In contrast, when the operation is determined not to be ended (NO in Step S107), the operation returns to Step S101.

Note that, although, in the example, a case where a charge of use is periodically calculated has been illustrated, this is not a limitation. For example, when the hospital 10 wants to refer to a charge of use at the present time, the charge of use may be calculated in response to a request from the hospital 10.

### 3. Second Embodiment

Next, a medical information system, a charge calculating device, and a charge calculating method according to a second embodiment will be described in detail with reference to the drawings. In the embodiment, a case where a charge of use in accordance with a working time (e.g., surgery time, analysis time, and the like) shortened by using the medical information system is calculated will be described.

Since a provision model and the medical information system according to the embodiment may be similar to the provision model and the medical information system described with reference to FIGS. 1 to 5 or 6 in the above-described first embodiment, detailed description thereof will be omitted here.

### 3.1 Example of Charge Calculating Operation

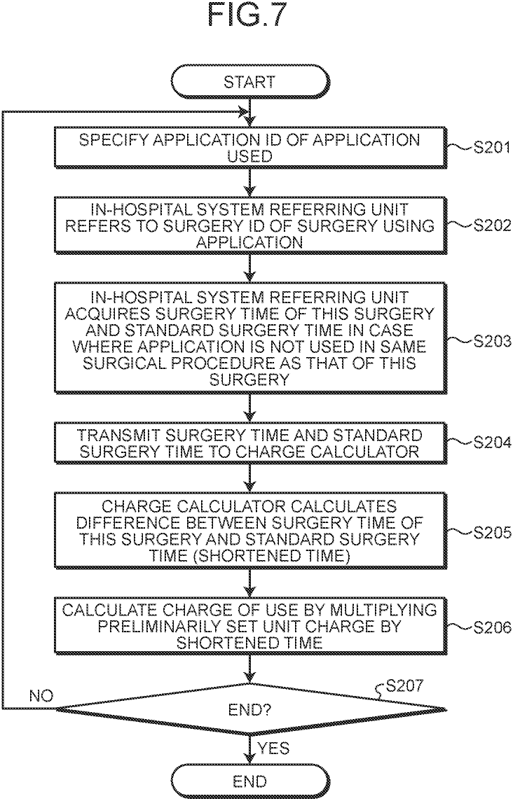

FIG. 7 is a flowchart illustrating one example of a charge calculating operation according to the embodiment. Note that, although, in the following description, a case where a charge generated when a medical information system is used during surgery is calculated will be illustrated similarly to the first embodiment, this is not a limitation. A charge generated when the medical information system is used for an image or a moving image recorded during surgery or sample data acquired at the time of parsing or analyzing a tissue sample can also be calculated.

As illustrated in FIG. 7, during surgery in a hospital 10, in the operation, similarly to Steps S101 and S102 in FIG. 6, a framework 301 specifies an application ID of an application 201 used during the surgery (Step S201), an in-hospital system referring unit 305 refers to an in-hospital system 103, the in-hospital system referring unit 305 refers to an in-hospital system 103, and thereby acquires a surgery ID of surgery using the application 201 corresponding to the specified application ID (Step S202).

Thereafter, when the surgery is completed, the in-hospital system referring unit 305 acquires a surgery time required for this surgery and a surgery time required for the same surgical procedure as this surgery in a case where the application 201 specified in Step S101 is not used (hereinafter, referred to as standard surgery time) by referring to the in-hospital system 103 (Step S203). Note that the standard surgery time is recorded in the in-hospital system 103. The standard surgery time provides an indication of a surgery time of each surgical procedure in the case where the medical information system is not used. The standard surgery time may be, for example, an average value of a plurality of surgeries of the same surgical procedure.

Then, the in-hospital system referring unit 305 transmits the acquired surgery time and standard surgery time to the charge calculator 303 (Step S204).

When receiving the surgery time and the standard surgery time, the charge calculator 303 calculates the difference therebetween, that is, a surgery time shortened by using the application 201 provided by the medical information system (shortened time) by subtracting the surgery time from the standard surgery time (Step S205).

Thereafter, the charge calculator 303 calculates a charge of use related to this surgery by, for example, multiplying a preliminarily set charge per unit shortened time (unit charge) by the shortened time calculated in Step S205 (Step S206). Note that the calculated charge of use may be recorded together with a date and time in the edge server 300, periodically aggregated, and charged to the hospital 10.

Thereafter, similarly to Step S107 in FIG. 6, the edge server 300 determines whether or not to end the operation (Step S207). When determining to end the operation (YES in Step S207), the edge server 300 ends the operation. In contrast, when the operation is determined not to be ended (NO in Step S207), the operation returns to Step S201.

Note that it is conceivable that the standard surgery time varies depending on a surgeon and medical staff (including the number of persons) in charge of the surgery, the type and number of medical devices used in the surgery, and the like. In that case, in the in-hospital system 103, the standard surgery time may be managed for each of a surgeon and medical staff (including the number of persons) in charge of the surgery, the type and number of medical devices used in the surgery, and the like.

### 4. Third Embodiment

Next, a medical information system, a charge calculating device, and a charge calculating method according to a third embodiment will be described in detail with reference to the drawings. In the embodiment, a case where a charge of use is calculated for each surgical procedure will be described.

Since a provision model and the medical information system according to the embodiment may be similar to the provision model and the medical information system described with reference to FIGS. 1 to 5 or 6 in the above-described first embodiment, detailed description thereof will be omitted here.

### 4.1 Example of Charge Calculating Operation

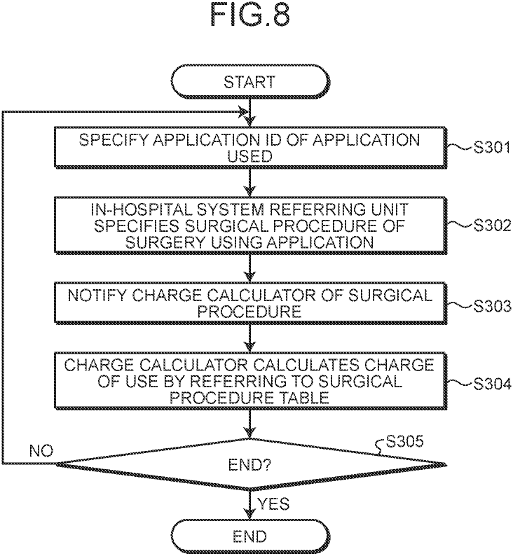

FIG. 8 is a flowchart illustrating one example of a charge calculating operation according to the embodiment. Note that, although, in the following description, a case where a charge generated when a medical information system is used during surgery is calculated will be illustrated similarly to the above-described embodiments, this is not a limitation. A charge generated when the medical information system is used for an image or a moving image recorded during surgery or sample data acquired at the time of parsing or analyzing a tissue sample can also be calculated.

As illustrated in FIG. 8, during surgery in a hospital 10, in the operation, similarly to Step S101 in FIG. 6, a framework 301 specifies an application ID of an application 201 used during the surgery (Step S301).

Next, the in-hospital system referring unit 305 specifies a surgical procedure of the surgery using the application 201 corresponding to the specified application ID by referring to the in-hospital system 103 (Step S302).

Then, the in-hospital system referring unit 305 notifies the charge calculator 303 of the specified surgical procedure (Step S303).

The charge calculator 303 calculates a charge of use of the application 201 by referring to the surgical procedure table 304 based on the surgical procedure about which a notification is given (Step S304). Note that the calculated charge of use may be recorded together with a date and time in the edge server 300, periodically aggregated, and charged to the hospital 10.

Thereafter, similarly to Step S107 in FIG. 6, the edge server 300 determines whether or not to end the operation (Step S305). When determining to end the operation (YES in Step S305), the edge server 300 ends the operation. In contrast, when the operation is determined not to be ended (NO in Step S305), the operation returns to Step S301.

### 5. Fourth Embodiment

Next, a medical information system, a charge calculating device, and a charge calculating method according to a fourth embodiment will be described in detail with reference to the drawings. In the embodiment, a case where a charge of use is calculated for each calculation cost of each application 201 will be described.

Since a provision model and the medical information system according to the embodiment may be similar to the provision model and the medical information system described with reference to FIGS. 1 to 5 or 6 in the above-described first embodiment, detailed description thereof will be omitted here.

### 5.1 Example of Charge Calculating Operation

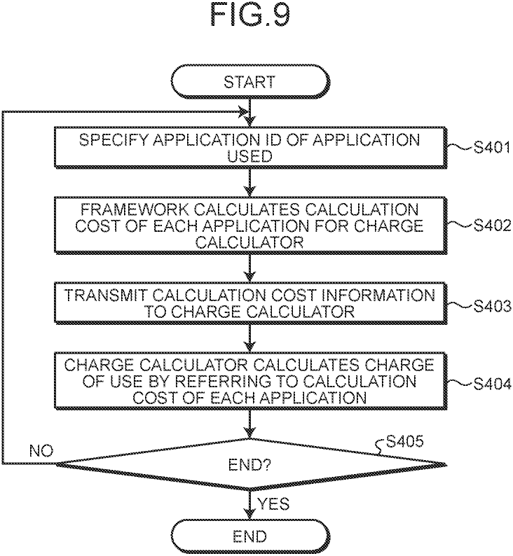

FIG. 9 is a flowchart illustrating one example of a charge calculating operation according to the embodiment. Note that, although, in the following description, a case where a charge generated when a medical information system is used during surgery is calculated will be illustrated similarly to the above-described embodiments, this is not a limitation. A charge generated when the medical information system is used for an image or a moving image recorded during surgery or sample data acquired at the time of parsing or analyzing a tissue sample can also be calculated.

As illustrated in FIG. 9, during surgery in a hospital 10, in the operation, similarly to Step S101 in FIG. 6, a framework 301 specifies an application ID of an application 201 used during the surgery (Step S401).

Next, the framework 301 calculates a calculation cost used by each application 201 for each application ID specified in Step S401 (Step S402). The calculation cost may be calculated based on, for example, CPU resources, memory resources, and processing time allocated to each application 201.

Then, the framework 301 transmits information on the calculated calculation cost (hereinafter, referred to as calculation cost information) to the charge calculator 303 (Step S403) .

The charge calculator 303 calculates a charge of use of each application 201 based on the received calculation cost information (Step S404). For example, the charge calculator 303 calculates the charge of use of each application 201 by multiplying a preliminarily set charge per calculation cost (unit charge) by the calculation cost. Note that the calculated charge of use may be recorded together with a date and time in the edge server 300, periodically aggregated, and charged to the hospital 10.

Thereafter, similarly to Step S107 in FIG. 6, the edge server 300 determines whether or not to end the operation (Step S405). When determining to end the operation (YES in Step S405), the edge server 300 ends the operation. In contrast, when the operation is determined not to be ended (NO in Step S405), the operation returns to Step S401.

### 6. Fifth Embodiment

Next, a medical information system, a charge calculating device, and a charge calculating method according to a fifth embodiment will be described in detail with reference to the drawings. In the embodiment, a case where a charge of use is calculated based on a substantial use time (hereinafter, also referred to as execution time) of each application 201 will be described.

Since a provision model and the medical information system according to the embodiment may be similar to the provision model and the medical information system described with reference to FIGS. 1 to 5 or 6 in the above-described first embodiment, detailed description thereof will be omitted here.

### 6.1 Example of Charge Calculating Operation

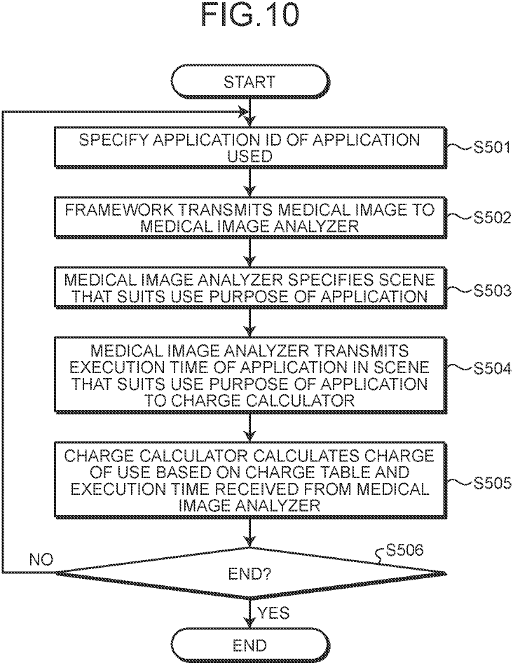

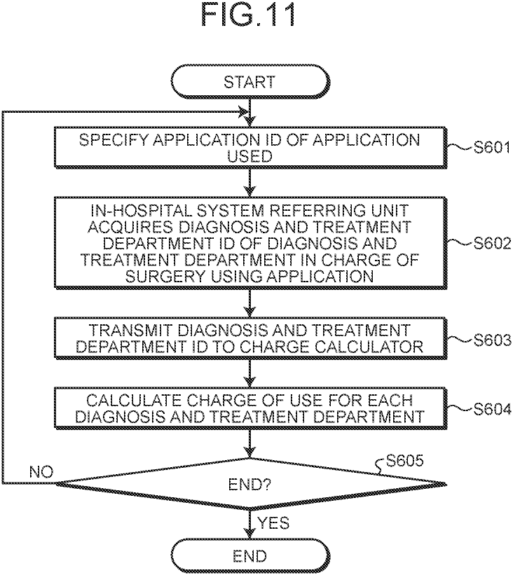

FIG. 10 is a flowchart illustrating one example of a charge calculating operation according to the embodiment. Note that, although, in the following description, a case where a charge generated when a medical information system is used during surgery is calculated will be illustrated similarly to the above-described embodiments, this is not a limitation. A charge generated when the medical information system is used for an image or a moving image recorded during surgery or sample data acquired at the time of parsing or analyzing a tissue sample can also be calculated.

As illustrated in FIG. 10, during surgery in a hospital 10, in the operation, similarly to Step S101 in FIG. 6, a framework 301 specifies an application ID of an application 201 used during the surgery (Step S501).

Next, the framework 301 transmits a medical image (e.g., surgical moving image obtained by capturing image of surgery) input from the medical device 101A to the medical image analyzer 302 (Step S502).

In contrast, the medical image analyzer 302 specifies a scene that suits a use purpose of the application 201 during the surgery by analyzing the medical image (Step S503). Note that the scene that suits a use purpose of the application 201 may be a scene in which use of the application 201 is scheduled or expected. The scene includes scenes of treatments of stopping bleeding from a blood vessel and cutting the blood vessel in a case of the application 201 that executes image processing of clearly displaying a region of the blood vessel in the medical image.

Then, the medical image analyzer 302 measures, from the specified scene, an execution time during which the application 201 that suits a use purpose of the scene in the scene is used, and transmits the measured execution time to the charge calculator 303 (Step S504).

For example, the charge calculator 303 calculates a charge of use of the application 201 in accordance with the execution time in the scene by referring to the charge table 306 (505). Note that the calculated charge of use may be recorded together with a date and time in the edge server 300, periodically aggregated, and charged to the hospital 10.

Thereafter, similarly to Step S107 in FIG. 6, the edge server 300 determines whether or not to end the operation (Step S506). When determining to end the operation (YES in Step S506), the edge server 300 ends the operation. In contrast, when the operation is determined not to be ended (NO in Step S506), the operation returns to Step S501.

### 7. Sixth Embodiment

Next, a medical information system, a charge calculating device, and a charge calculating method according to a sixth embodiment will be described in detail with reference to the drawings. In the embodiment, a case where a charge of use of each application 201 is calculated for each diagnosis and treatment department will be described.

Since a provision model and the medical information system according to the embodiment may be similar to the provision model and the medical information system described with reference to FIGS. 1 to 5 or 6 in the above-described first embodiment, detailed description thereof will be omitted here.

### 7.1 Example of Charge Calculating Operation

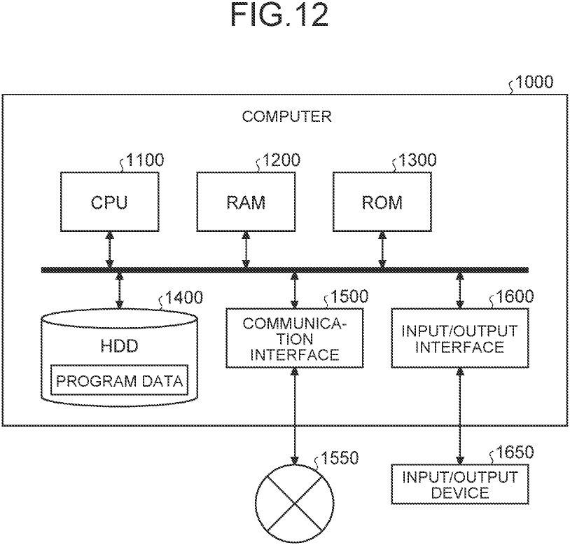

FIG. 11 is a flowchart illustrating one example of a charge calculating operation according to the embodiment. Note that, although, in the following description, a case where a charge generated when a medical information system is used during surgery is calculated will be illustrated similarly to the above-described embodiments, this is not a limitation. A charge generated when the medical information system is used for an image or a moving image recorded during surgery or sample data acquired at the time of parsing or analyzing a tissue sample can also be calculated.

As illustrated in FIG. 11, during surgery in a hospital 10, in the operation, similarly to Step S101 in FIG. 6, a framework 301 specifies an application ID of an application 201 used during the surgery (Step S601).

Next, the in-hospital system referring unit 305 acquires a diagnosis and treatment department ID for specifying a diagnosis and treatment department in charge of the above-described surgery by referring to the in-hospital system 103 (Step S602).

Then, the in-hospital system referring unit 305 transmits the specified diagnosis and treatment department ID to the charge calculator 303 (Step S603).

For example, the charge calculator 303 calculates a charge of use of the application 201 for each diagnosis and treatment department by referring to the charge table 306 (604). Note that the calculated charge of use may be recorded together with a date and time in the edge server 300, periodically aggregated, and charged to the hospital 10.

Thereafter, similarly to Step S107 in FIG. 6, the edge server 300 determines whether or not to end the operation (Step S605). When determining to end the operation (YES in Step S605), the edge server 300 ends the operation. In contrast, when the operation is determined not to be ended (NO in Step S605), the operation returns to Step S501.

### 8. Other Embodiments

Note that the charge calculating operations according to the above-described embodiments may be appropriately combined. A charge of use of an application 201 in accordance with an execution time in a scene may be calculated in accordance with a surgical procedure of surgery by combining the charge calculating operation according to the fifth embodiment with the charge calculating operation according to the third embodiment, for example. Furthermore, a charge of use in accordance with a surgery shortened time and a calculation cost of each application 201 may be calculated by combining the charge calculating operation according to the second embodiment with the charge calculating operation according to the fourth embodiment, for example. Moreover, a charge of use in accordance with the number of times of use of each application 201 may be calculated for each surgical procedure by combining the charge calculating operation according to the first embodiment with the charge calculating operation according to the third embodiment, for example.

Furthermore, in the above-described embodiments, a charge calculator may calculate an amount of use based on a situation of use of an application installed in a converter connected to a medical device in addition to an application installed in an edge server 300. The converter is, for example, an internet protocol (IP) converter that converts a signal output from the medical device into an IP signal. A signal converted by an input-side converter connected to the medical device is transmitted to the edge server 300 or an output-side converter via a switcher, and is output to a medical device (e.g., display device) connected to the output-side converter. In this case, an input signal from the medical device can be processed by an application preliminarily installed in the input-side converter or the output-side converter. The charge calculator determines a situation of use of either or both of an application of the edge server and an application of the converter, and calculates a charge of use. This enables distributed processing. In the distributed processing, processing in which real-time property is important is performed by the application of the converter, and processing with a large calculation amount is performed by the edge server.

### 9. Conclusion

As described above, according to the above-described embodiments, a charge of use of the application 201 can be calculated in accordance with the number of times of surgeries using the application 201, a surgery time (reduction in surgery time), whether or not a medical insurance is applied, a use calculation cost, a used diagnosis and treatment department, and the like.

Furthermore, according to the above-described embodiments, a charge of use can be calculated on the edge server 300, an in-hospital server outside the edge server, or a cloud server. When the charge of use is calculated outside the edge server 300, the edge server 300 transmits information necessary for calculating the charge of use to the charge calculator 303.

According to the above-described embodiments, the configuration as described above allows the application 201 to be flexibly changed even after introduction of a system, so that costs for a system change can be inhibited.

Furthermore, according to the above-described embodiments, an appropriate charge of use can be calculated from a situation of use of the application 201 in a surgery scene. This allows a user to flexibly select the necessary application 201 from many options provided by the application provider 20.

Moreover, according to the above-described embodiments, it is unnecessary to frequently stop the application 201 during surgery for the purpose of inhibiting a charge of use. Thus, an appropriate charge of use can be calculated while preventing progress of the surgery from being disturbed.

### 10. Hardware Configuration

The edge server 300 and/or the cloud server/in-hospital server 320 according to the embodiments, the variations, and the application examples thereof described above can be implemented by a computer 1000 having a configuration as illustrated in FIG. 12, for example. FIG. 12 is a hardware configuration diagram illustrating one example of the computer 1000 that implements the functions of the edge server 300 and/or the cloud server/in-hospital server 320. The computer 1000 includes a CPU 1100, a RAM 1200, a read only memory (ROM) 1300, a hard disk drive (HDD) 1400, a communication interface 1500, and an input/output interface 1600. Each unit of the computer 1000 is connected by a bus 1050.

The CPU 1100 operates based on a program stored in the ROM 1300 or the HDD 1400, and controls each unit. For example, the CPU 1100 develops a program stored in the ROM 1300 or the HDD 1400 on the RAM 1200, and executes processing corresponding to various programs.

The ROM 1300 stores a boot program such as a basic input output system (BIOS) executed by the CPU 1100 at the time when the computer 1000 is started, a program depending on the hardware of the computer 1000, and the like.

The HDD 1400 is a computer-readable recording medium that non-transiently records a program executed by the CPU 1100, data used by the program, and the like. Specifically, the HDD 1400 is a recording medium that records a projection control program according to the present disclosure. The projection control program is one example of a program data 1450.

The communication interface 1500 is used for connecting the computer 1000 to an external network 1550 (e.g., Internet). For example, the CPU 1100 receives data from another device and transmits data generated by the CPU 1100 to another device via the communication interface 1500.

The input/output interface 1600 has a configuration including the above-described I/F unit 18, and connects an input/output device 1650 with the computer 1000. For example, the CPU 1100 receives data from an input device such as a keyboard and a mouse via the input/output interface 1600. Furthermore, the CPU 1100 transmits data to an output device such as a display, a speaker, and a printer via the input/output interface 1600. Furthermore, the input/output interface 1600 may function as a medium interface that reads a program and the like recorded in a predetermined recording medium (medium). The medium includes, for example, an optical recording medium such as a digital versatile disc (DVD) and a phase change rewritable disk (PD), a magneto-optical recording medium such as a magneto-optical disk (MO), a tape medium, a magnetic recording medium, a semiconductor memory, and the like.

For example, when the computer 1000 functions as the edge server 300 and/or the cloud server/in-hospital server 320 according to the above-described embodiments, the CPU 1100 of the computer 1000 implements the functions of the framework 301, the medical image analyzer 302, the charge calculator 303, the in-hospital system referring unit 305, the charge-of-use calculation information transmitter 311, and the like by executing a program loaded on the RAM 1200. Furthermore, the HDD 1400 stores a program and the like according to the present disclosure. Note that the CPU 1100 reads the program data 1450 from the HDD 1400 and executes the program data 1450. In another example, the CPU 1100 may acquire these programs from another device via the external network 1550.

Although the preferred embodiments of the present disclosure have been described in detail with reference to the accompanying drawings, the technical scope of the present disclosure is not limited to such examples. It is obvious that a person having ordinary knowledge in the technical field of the present disclosure can conceive various changes or modifications within the scope of the technical idea described in claims, and it is naturally understood that these changes or modifications also belong to the technical scope of the present disclosure.

Furthermore, the effects described in the present specification are merely illustrative or exemplary ones, and are not limitations. That is, the technology according to the present disclosure can exhibit other effects obvious to those skilled in the art from the description of the present specification together with or instead of the above-described effects.

Note that the following configurations also belong to the technical scope of the present disclosure.

A medical information system including an edge server connected to a medical device that acquires a medical image during surgery,
wherein the edge server includes:
   a framework including one or more applications that process the medical image; and
   a charge calculator that calculates a charge in accordance with a situation of use of the one or more applications for the medical image.

The medical information system according to (1), further including a charge table that manages a charge for each of numbers of times of use for each of the one or more applications,

wherein the charge calculator calculates the charge by referring to the charge table based on a number of times of use of each of the one or more applications in the surgery.

The medical information system according to (1) or (2), further including an in-hospital server referring unit that acquires information on the surgery from an in-hospital server installed in a hospital,

wherein the charge calculator calculates the charge based on a difference between a standard surgery time taken for a same surgical procedure as that of the surgery included in the information on the surgery acquired by the in-hospital server referring unit and a surgery time of the surgery.

The medical information system according to any one of (1) to (3),
wherein the framework transmits information on a calculation cost of each of the one or more applications for the medical image to the charge calculator, and
the charge calculator calculates the charge based on the calculation cost of each of the one or more applications.

The medical information system according to any one of (1) to (4), further including a medical image analyzer that specifies a scene that suits a use purpose of each of the one or more applications during the surgery from the medical image,

wherein the charge calculator calculates the charge based on an execution time of the one or more applications in the scene.

The medical information system according to any one of (1) to (5), further including an in-hospital server referring unit that acquires information on the surgery from an in-hospital server installed in a hospital,

wherein the charge calculator calculates the charge for a diagnosis and treatment department in charge of the surgery included in the information on the surgery acquired by the in-hospital server referring unit.

The medical information system according to any one of (1) to (6), wherein the charge calculator periodically calculates the charge.

The medical information system according to any one of (1) to (7), wherein the charge calculator adds a charge in accordance with a situation of use of an application installed in a converter connected to the medical device for the medical image.

A charge calculating device including:
a framework including one or more applications that process a medical image acquired during surgery; and
a charge calculator that calculates a charge in accordance with a situation of use of the one or more applications for the medical image.

A charge calculating method including calculating a charge in accordance with a situation of use of one or more applications that process a medical image acquired during surgery for the medical image.

Reference Signs List

| | |
|---|---|
| 10 | HOSPITAL |
| 20 | APPLICATION PROVIDER |
| 30 | EDGE SERVER PROVIDER |
| 101, 101B | MEDICAL DEVICE |
| 103 | IN-HOSPITAL SYSTEM |
| 201a, 201b | ,... APPLICATION |
| 300 | EDGE SERVER |
| 301 | FRAMEWORK |
| 302 | MEDICAL IMAGE ANALYZER |
| 303 | CHARGE CALCULATOR |
| 304 | SURGICAL PROCEDURE TABLE |
| 305 | IN-HOSPITAL SYSTEM REFERRING UNIT |
| 306 | CHARGE TABLE |
| 311 | CHARGE-OF-USE CALCULATION INFORMATION TRANSMITTER |
| 320 | CLOUD SERVER/IN-HOSPITAL SERVER |

1. A medical information system including an edge server connected to a medical device that acquires a medical image during surgery,
wherein the edge server includes:
a framework including one or more applications that process the medical image; and
a charge calculator that calculates a charge in accordance with a situation of use of the one or more applications for the medical image.

2. The medical information system according to claim 1, further including a charge table that manages a charge for each of numbers of times of use for each of the one or more applications,
wherein the charge calculator calculates the charge by referring to the charge table based on a number of times of use of each of the one or more applications in the surgery.

3. The medical information system according to claim 1, further including an in-hospital server referring unit that acquires information on the surgery from an in-hospital server installed in a hospital,
wherein the charge calculator calculates the charge based on a difference between a standard surgery time taken for a same surgical procedure as that of the surgery included in the information on the surgery acquired by the in-hospital server referring unit and a surgery time of the surgery.

4. The medical information system according to claim 1,
wherein the framework transmits information on a calculation cost of each of the one or more applications for the medical image to the charge calculator, and
the charge calculator calculates the charge based on the calculation cost of each of the one or more applications.

5. The medical information system according to claim 1, further including a medical image analyzer that specifies a scene that suits a use purpose of each of the one or more applications during the surgery from the medical image,
wherein the charge calculator calculates the charge based on an execution time of the one or more applications in the scene.

6. The medical information system according to claim 1, further including an in-hospital server referring unit that acquires information on the surgery from an in-hospital server installed in a hospital,
wherein the charge calculator calculates the charge for a diagnosis and treatment department in charge of the surgery included in the information on the surgery acquired by the in-hospital server referring unit.

7. The medical information system according to claim 1, wherein the charge calculator periodically calculates the charge.

8. The medical information system according to claim 1, wherein the charge calculator adds a charge in accordance with a situation of use of an application installed in a converter connected to the medical device for the medical image.

9. A charge calculating device including:
a framework including one or more applications that process a medical image acquired during surgery; and
a charge calculator that calculates a charge in accordance with a situation of use of the one or more applications for the medical image.

10. A charge calculating method including calculating a charge in accordance with a situation of use of one or more applications that process a medical image acquired during surgery for the medical image.
